(19) United States
(12) Patent Application Publication
(10) Pub. No.: US 2024/0409537 A1
(43) Pub. Date: Dec. 12, 2024

(54) Crystal Form of AMG510 Compound, and Preparation Method Thereof and Use Thereof

(71) Applicants: BIRDO (SHANGHAI) PHARMACEUTICAL R&D CO., LTD, Shanghai (CN); BIRDO (SHANGHAI) PHARMATECH CO., LTD, Shanghai (CN)

(72) Inventors: Lei WANG, Shanghai (CN); Zhaohui YANG, Shanghai (CN); Zisheng ZHENG, Shanghai (CN); Jiazhi LI, Shanghai (CN)

(73) Assignees: BIRDO (SHANGHAI) PHARMACEUTICAL R&D CO., LTD, Shanghai (CN); BIRDO (SHANGHAI) PHARMATECH CO., LTD, Shanghai (CN)

(21) Appl. No.: 18/692,837

(22) PCT Filed: Aug. 9, 2022
(86) PCT No.: PCT/CN2022/111034

§ 371 Date: Mar. 18, 2024

(30) Foreign Application Priority Data
Sep. 18, 2021 (CN) 202111098878.X
Mar. 11, 2022 (CN) 202210243070.4

(51) Int. Cl. C07D 471/04; A61K 31/519

(52) U.S. Cl. CPC C07D 471/04; A61K 31/519

(57) ABSTRACT

A crystal form of a compound I and a preparation method thereof, a pharmaceutical composition containing the crystal form, and the use of the crystal form in the preparation of a drug for treating related diseases caused by KRAS G12C gene mutation are provided. The crystal form DCIII of the compound I provided by the present invention has important value for the development of the drug in the future.

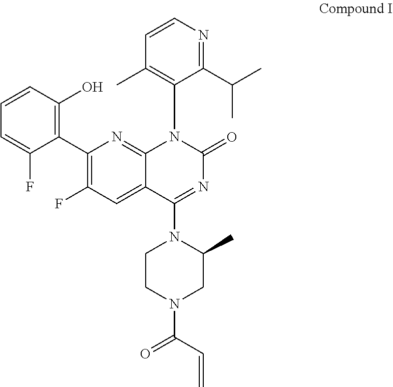 [SMILES: C=CC(=O)N1CCN(c2nc(=O)n(-c3c(C)ccnc3C(C)C)c3nc(-c4c(O)cccc4F)c(F)cc23)[C@@H](C)C1]

### CROSS REFERENCE TO THE RELATED APPLICATIONS

This application is the national phase entry of International Application No. PCT/CN2022/111034, filed on Aug. 9, 2022, which is based upon and claims priority to Chinese Patent Application No. 202210243070.4, filed on Mar. 11, 2022, and Chinese Patent Application No. 202111098878.X, filed on Sep. 18, 2021, the entire contents of which are incorporated herein by reference.

### TECHNICAL FIELD

The present invention relates to the field of medicinal chemistry. The present invention particularly relates to a crystal form of an AMG510 compound, and a preparation method thereof and use thereof.

### BACKGROUND

A protein coded by a KRAS gene is a signal transduction protein in an intracellular signal transduction pathway, which has an important impact on the growth, survival, differentiation and other functions of cells. When the KRAS gene is mutated, a normal RAS protein cannot be produced, resulting in intracellular signal transduction disorder and uncontrolled cell proliferation, thus leading to canceration. KRAS G12C mutations typically occur in about 13% of lung cancer patients, 3% of colorectal and appendiceal cancer patients, and 1% to 3% of other solid tumor patients. KRAS is a member of the RAS family of oncogenes, and mutations of KRAS may induce constitutive signal transduction, leading to tumor cell growth, proliferation, invasion, and metastasis.

On May 28, 2021, the FDA approved Amgen's first KRAS-targeted drug Lumakras (i.e., sotorasib, formerly known as AMG510), for non-small cell lung cancer (NSCLC) harboring the KRAS G12C mutation, ending the history of this “strongest” carcinogenic mutation as no cure. AMG510 is an oral KRAS G12C small molecule inhibitor targeting oncogenic KRAS substitution mutation G12C, achieving favorable anti-tumor activity.

The chemical name of the AMG510 compound is 6-fluoro-7-(2-fluoro-6-hydroxyphenyl)-1-(4-methyl-2-propane-2-ylpyridin-3-yl)-4-[(2S)-2-methyl-4-propyl-2-enoylpiperazin-1-yl]pyridine[2,3-d]pyrimidin-2-one (hereinafter referred to as “Compound I”), having the following structural formula:

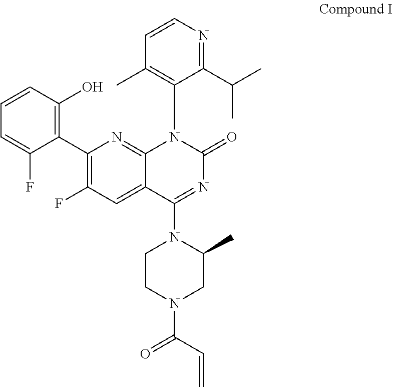 [SMILES: C=CC(=O)N1CCN(c2nc(=O)n(-c3c(C)ccnc3C(C)C)c3nc(-c4c(O)cccc4F)c(F)cc23)[C@@H](C)C1]

A crystal form is a solid form of solid molecules of a compound arranged in a microscopic three-dimensional structure in a long-term orderly manner to form a crystal lattice. The drug heteromorphism refers to a phenomenon of two or more different crystal forms of solid drug molecules. Because different crystal forms have different physicochemical properties, different crystal forms of solid drug molecules may have different dissolution and absorption in vivo, which will further affect the clinical efficacy and safety of drugs to a certain extent, and especially for poorly soluble solid drugs, will have a greater impact on bioavailability. Therefore, crystal forms of drugs are of an important part of the research and development process of solid-state drugs, and also of an important part of drug quality control.

The innovator patents US20200369662 and WO2020236947 reported anhydrous crystal forms I to III, a hydrate crystal form I, as well as a THE solvate, a MeCN solvate, a MEK solvate, a DCM solvate, an acetone solvate, a methanol solvate, an isopropanol solvate and an ethanol solvate. In the description of this patent document, the anhydrous crystal form I is the most thermodynamically stable crystal form. The anhydrous crystal form I has the disadvantage of low solubility although it has good stability. Both the anhydrous crystal form II and the anhydrous crystal form III will be transformed into the anhydrous crystal form I, and have hygroscopicity, but have disadvantages in pharmaceutical development. In addition, a preparation process of the hydrate crystal form took 13 days, resulting in low production efficiency. The rest of the solvates contain organic solvents, which have greater toxic and side effects, and thus cannot be used as pharmaceutical crystal forms.

In summary, there is an urgent need for a new crystal form of AMG510 with good solubility and stability in this field to meet the bioavailability of drugs, which is suitable for industrial development and meets the pharmaceutical development in terms of comprehensive properties.

The inventors of the present application have accidentally discovered different crystal forms of a compound I provided by the present invention, which have advantages in terms of physicochemical properties, preparation processing properties and bioavailability, such as at least one aspect of a melting point, solubility, hygroscopicity, a purification effect, stability, adhesion, compressibility, fluidity, in vitro and in vitro dissolution and bioavailability, provide a better choice for the development of drugs containing the compound I, and thus are of great significance.

### SUMMARY

A main objective of the present invention is to provide a new crystal form of a compound I, and a preparation method and use thereof.

According to the objective of the present invention, the present invention provides a crystal form of a compound I.

Further, the crystal form of the compound I provided by the present invention may be a crystal form DCIII (hereinafter referred to as a crystal form DCIII).

In an aspect, with the use of Cu—Kα radiation, a characteristic peak appears at one, two or three of diffraction angles 2theta having values of 6.3°±0.2°, 9.0° 0.2° and 14.8°+0.2° in an X-ray powder diffraction pattern of the crystal form DCIII.

Further, with the use of Cu—Kα radiation, a characteristic peak appears at one, two or three of diffraction angles 2theta having values of 17.8°+0.2°, 12.7°+0.2° and 16.5°+0.2° in the X-ray powder diffraction pattern of the crystal form DCIII. Preferably, a characteristic peak appears at three diffraction angles 2theta further having values of 17.8°+0.2°, 12.7°+0.2° and 16.5°+0.2° in the X-ray powder diffraction pattern of the crystal form DCIII.

Further, with the use of Cu—Kα radiation, a characteristic peak appears at one, two or three of diffraction angles 2theta having values of 12.2°+0.2°, 13.4°+0.2° and 20.3°+0.2° in the X-ray powder diffraction pattern of the crystal form DCIII. Preferably, a characteristic peak appears at three diffraction angles 2theta having values of 12.2°+0.2°, 13.4°+0.2° and 20.3°+0.2° in the X-ray powder diffraction pattern of the crystal form DCIII.

In another aspect, with the use of Cu—Kα radiation, a characteristic peak appears at one, or two, or three, or four, or five, or six, or seven, or eight, or nine, or ten, or eleven of diffraction angles 2theta further having values of 6.3°+0.2°, 9.0°+0.2°, 12.2°+0.2°, 12.7°+0.2°, 13.4°+0.2°, 14.8°+0.2°, 16.5°+0.2°, 17.8°+0.2°, 20.3°+0.2°, 23.2°+0.2°, and 27.8°+0.2° in the X-ray powder diffraction pattern of the crystal form DCIII.

Non-limitatively, the X-ray powder diffraction pattern of the crystal form DCIII is basically shown in FIG. 3.

Non-limitatively, an endothermic peak of the crystal form DCIII begins to appear near 289°, and a differential scanning calorimetry analysis diagram is basically shown in FIG. 4.

Non-limitatively, the crystal form DCIII is an anhydrous crystal form.

According to the objective of the present invention, the present invention further provides a preparation method of the crystal form DCIII. The method includes:

### Method I:

weighing a certain amount of compound I; adding a certain amount of organic solvent or a mixed solvent of organic solvents; stirring, and performing recrystallization at a certain temperature; and centrifuging for solid separation to obtain the crystal form DCIII.

Specifically, a certain amount of compound I is weighed and added to a glass bottle, added with a certain amount of organic solvent or a mixed solvent of organic solvents (e.g., ethers, or esters, or alkanes, or nitriles, or alcohols, or a mixture of two of the above), shaken fully, recrystallized at a certain temperature, and centrifuged for solid separation to obtain the crystal form DCIII.

Further, ethers may be methyl tert-butyl ether; alkanes may be n-hexane; and alcohols may be n-butanol.

Further, in the case of stirring at 5° C.±2° C., the solid is separated by centrifugation, and dried at 30° C.±2° C. to obtain the crystal form DCIII.

In some examples, a certain amount of compound I is weighed, and added with a certain amount of alcohols or a mixed solvent containing an alcohol solvent; and a sample is dissolved and clarified, then added with an alkane reagent, stirred at a certain temperature for a period of time, and centrifuged for solid separation to obtain the crystal form DCIII.

Specifically, a certain amount of compound I is weighed and added to a glass bottle, added with a certain amount of alcohols or a mixed solvent containing an alcohol solvent; and a sample is dissolved and clarified, then added with an alkane reagent, stirred at a certain temperature for a period of time, and centrifuged for solid separation to obtain the crystal form DCIII.

Further, the alcohols are n-butanol; and the mixed solvent containing the alcohol solvent is a methyl tert-butyl ether mixed solvent containing n-butanol or an n-hexane mixed solvent containing n-butanol.

Further, in the case of stirring at 5° C.±2° C., the solid is separated by centrifugation to obtain the crystal form DCIII.

### Method II:

weighing a certain amount of compound I; adding a certain amount of water to form a suspension; stirring for a period of time; separating a solid, and heating the solid to high temperature; and collecting the solid to obtain the crystal form DCIII.

Specifically, a certain amount of compound I is weighed and added to a vial, added with a certain amount of water to form a suspension, and stirred for a period of time; a solid is then separated, and heated to high temperature; and the solid is collected to obtain the crystal form DCIII.

Further, the selected high temperature is preferably 190° C. to 235° C., more preferably, 220° C.

The crystal form DCIII provided by the present invention has the following beneficial effects.

1) Compared with the prior art, the crystal form DCIII of the present invention has higher solubility.

Compared with the prior art, the crystal form DCIII of the present invention has higher solubility in simulated gastric fluid (SGF), fasted state simulated intestinal fluid (FaSSIF), fed state simulated intestinal fluid (FeSSIF) and pure water. At 1 h, 4 h and 24 h, the solubility of the crystal form DCIII of the present invention is 2 to 3 times that of an anhydrous crystal form reported in the prior art US20200369662A1. Higher solubility is conducive to improving the absorption of drugs in the human body, improving the bioavailability of drugs, and achieving better therapeutic effects with less drug loading. In addition, under the premise of ensuring the efficacy of drugs, the toxic and side effects of drugs can be reduced by reducing the drug loading, and the safety of drug use is improved, which is of great clinical significance.

2) The crystal form DCIII provided by the present invention has good stability.

The crystal form DCIII of the present invention is placed airtightly for 1 month respectively under the conditions of 25° C./60% RH (relative humidity), 40° C./75% RH, and 60° C./75% RH, and the crystal form remains unchanged, indicating that the crystal form DCIII has good physical stability. Especially under an acceleration condition of 40° C./75% RH, and a high temperature and humidity condition of 60° C./75% RH, the crystal form is still stable after being placed for one month, and no crystal transformation occurs, further indicating that the crystal form DCIII still has good physical stability even under the high temperature and high humidity condition, which ensures that a drug is not prone to crystal transformation in the subsequent technologies, as well as production and transportation processes. In addition, the chemical purity of the crystal form DCIII does not change before and after placement under a condition of 25° C./60% RH (relative humidity), and the purity remains above 99%, indicating that the crystal form DCIII has good chemical stability. In addition, even under the acceleration condition of 40° C./75% RH and the high temperature and humidity condition of 60° C./75% RH, the chemical purity still does not present a significant decrease, further indicating that the crystal form DCIII has good chemical stability. Good physiochemical stability is of great significance to ensure that the quality of the drugs can be maintained stable in the subsequent preparation development and technical production process, as well as in the process of drug production and transportation, and to ensure the quality and efficacy of the drugs.

Furthermore, the crystal form DCIII provided by the present invention has good mechanical stability. The crystal form DCIII has no crystal transformation before and after grinding, and no significant decrease observed in the crystallinity of a sample, indicating that the DCIII has good mechanical stability. Good mechanical stability can ensure that the sample will not be prone to crystal transformation due to external forces such as mechanical grinding and crushing in the later preparation process, which reduces the risk of crystal transformation in the preparation process and improves the developability of the preparation process.

Crystal form stabilization is of great significance for drug development. If crystal transformation occurs, it will directly affect the solubility of drugs and thus the bioavailability of the drugs, thereby changing the efficacy of the drugs. Good chemical stability may ensure that the drugs produce almost no new impurities or the impurity content barely increases during storage, thus ensuring the safety of the drugs. Good mechanical stability can also improve the resistance of the drugs to mechanical force damage during the preparation process and reduce the risk of crystal transformation. Therefore, good physicochemical stability and good mechanical stability of the crystal form DCIII provide a guarantee for the production and development of follow-up drugs, and thus the crystal form DCIII has a high industrial development value.

Further, the crystal form DCIII of the present invention further has the following beneficial effects.

1) The crystal form DCIII of the present invention has low hygroscopicity.

According to the pharmacopoeia (9103 Drug Hygroscopicity Experiment Guiding Principles, 2020 edition of General Principles, Chinese Pharmacopoeia, experimental conditions: 25±1° C., 80% relative humidity) method, the hygroscopicity of the crystal form DCIII of the present invention is investigated, and the results show that the hygroscopic weight gain of the crystal form DCIII is increased by 0.6%. In addition, according to the description of hygroscopicity characteristics and the definition (9103 Drug Hygroscopicity Experiment Guiding Principles, 2020 edition of General Principles, Chinese Pharmacopoeia, experimental conditions: 25±1° C., 80% relative humidity) principle of the hygroscopic weight gain, the weight gain of the crystal form DCIII has a range of less than 2.0% but not less than 0.2%, showing slight hygroscopicity. This result shows that the crystal form DCIII has low hygroscopicity. The low hygroscopicity can ensure that the sample can maintain low hygroscopic weight gain without a deliquescent phenomenon during post-production, processing, storage and transportation, thereby ensuring stable quality of the drugs.

2) The crystal form DCIII of the present invention has a good purification effect, and is very suitable for industrial production.

After recrystallization to prepare the crystal form DCIII of the present invention, the chemical purity of the sample is increased from 98.4% to 99.1%, indicating that the crystal form DCIII has good purification and impurity removal effects, which not only improves the quality and safety of the drugs, but also is very suitable for industrial large-scale production.

According to the objective of the present invention, the present invention further provides a pharmaceutical composition. The pharmaceutical composition contains a therapeutically effective amount of the crystal form DCIII of the compound I and a pharmaceutically acceptable carrier or excipient.

Further, the present invention provides use of the crystal form DCIII of the compound I in the preparation of a KRAS G12C inhibitor drug.

Furthermore, the present invention provides use of the crystal form DCIII of the compound I in the preparation of a drug for treatment non-small cell lung cancer, colorectal cancer, or appendiceal cancer.

In the present invention, the “stirring” is completed by a conventional method in the art, such as magnetic stirring or mechanical stirring, and a stirring speed is 50-1800 rpm, wherein the magnetic stirring is preferably 300-900 rpm, and the mechanical stirring is preferably 100-300 rpm.

The “separating” is completed by the conventional method in the art, such as centrifugation or filtration, and the operation of “centrifugation” is to place a sample to be separated in a centrifuge tube, centrifuge at a rate of 10,000 rpm, until all the solid sinks to the bottom of the centrifuge tube.

The “drying” may be carried out at room temperature or higher. A drying temperature is room temperature to about 50° C., or to 40° C. The drying time may be 2 to 48 h, or overnight. Drying takes place in a fume hood, a blast oven or a vacuum oven.

In the present invention, “crystal” or “polymorphic form” refers to a solid that is confirmed by characterization with the X-ray powder diffraction pattern. Those skilled in the art can understand that the physicochemical properties discussed here can be characterized, where an experimental error depends on conditions of an instrument, the preparation of a sample, and the purity of the sample. In particular, it is known to those skilled in the art that the X-ray powder diffraction pattern usually changes depending on different conditions of instruments. In particular, it should be noted that the relative intensity of a diffraction peak in the X-ray powder diffraction pattern may also vary with the experimental conditions, so an order of the intensities of the diffraction peaks cannot be used as a sole or decisive factor. In fact, the relative intensities of the diffraction peaks in the X-ray powder diffraction pattern are related to preferred orientations of crystals, and the intensities of the diffraction peaks shown in the present invention are illustrative rather than for absolute comparison. In addition, an experimental error at the diffraction peak position is generally 5% or less, and errors at these positions should also be taken into account. An error of ±0.2 is usually allowed. In addition, due to the influence of experimental factors such as sample thickness, the overall shift of a diffraction peak angle will occur, and a certain shift is usually allowed. Therefore, those skilled in the art may understand that the X-ray powder diffraction pattern of the crystal form of the present invention is not necessarily completely consistent with the X-ray powder diffraction pattern in the example involved herein, and any crystal form with the same or similar X-ray powder diffraction pattern with the characteristic peaks in these patterns falls within the scope of the present invention.

Those skilled in the art can compare the X-ray powder diffraction patterns listed in the present invention with an X-ray powder diffraction pattern of an unknown crystal form to confirm whether two sets of patterns reflect the same or different crystal forms.

In some embodiments, the crystal form DCIII of the present invention is pure, and is basically not doped with other crystal form. In the present invention, “basically not”, when used to refer to the new crystal form, means that this crystal form contains less than 20% (weight) of other crystal forms, in particular to less than 10% (weight) of other crystal forms, particularly less than 5% (weight) of other crystal forms, and particularly less than 1% (weight) of other crystal forms. The term “about” in the present invention, when used to refer to measurable numerical values, such as mass, time and temperature, means that there is a certain range of fluctuations around a specific value, which may be ±10%, ±5%, ±1%, ±0.5% or ±0.1%.

In the present invention, “° C.” is used as a unit of temperature, which should be understood as degrees Celsius.

### BRIEF DESCRIPTION OF THE DRAWINGS

FIG. 1 is an XRPD diagram of a crystal form DCIII obtained according to Example 1a;

FIG. 2 is an DSC diagram of a crystal form DCIII obtained according to Example 1a;

FIG. 3 is an XRPD diagram of a crystal form DCIII obtained according to Example 1b;

FIG. 4 is an DSC diagram of a crystal form DCIII obtained according to Example 1b;

FIG. 5 is an XRPD diagram of a crystal form DCIII obtained according to Example 1c;

FIG. 6 is an XRPD overlay after stable placement of a crystal form DCIII (from top to bottom: onset crystal form DCIII, the crystal form DCIII is placed at 25° C./60% RH for 1 month, at 40° C./75% RH for 1 month, and at 60° C./75% RH for 1 month); and

FIG. 7 is an XRPD overlay before and after grinding of a crystal form DCIII (the upper diagram is an XRPD diagram before grinding, and the lower diagram is an XRPD diagram after grinding).

### DETAILED DESCRIPTION OF THE EMBODIMENTS

The present invention is described in detail in conjunction with the following examples, which are used to detail a preparation method and a use method of a crystal form of the present invention. It is obvious to those skilled in the art that many changes to both materials and methods can be made without departing from the scope of the present invention.

The abbreviations used in the present invention were explained as follows:
XRPD: X-ray powder diffraction
DSC: differential scanning calorimetry

### Instruments and Methods Used to Collect Data:

an X-ray powder diffraction pattern described in the present invention was collected on a Bruker D2 PHASER X-ray powder diffractometer. Method parameters of X-ray powder diffraction described in the present invention were as follows:
X-ray light source: Cu Kα
Ka1 (A): 1.54060; Ka2 (A) 1.54439
Ka2/Ka1 intensity ratio: 0.50
voltage: 30 kilovolt (kV)
current: 10 milliamperes (mA)
scanning range: from 3.0 to 40.0°

A DSC diagram described in the present invention was collected on METTLER DSC3, and method parameters of DSC were as follows:
scanning rate: 10° C./min
protective gas: nitrogen

Unless otherwise specified, the following examples were all operated at room temperature, and the “room temperature” was not a specific temperature value, but refers to a temperature range of 10-30° C.

According to the present invention, the compound I and/or its salts as raw materials include, but are not limited to, a solid form (crystalline or amorphous), an oily form, a liquid form and a solution. Preferably, the compound I and/or its salts as the raw materials were in solid forms.

The compound I used in the following example may be prepared according to a method recorded in the literature.

### Example 1: Preparation Method of Crystal Form DCIII

### Example 1a

1 g of compound I was weighed and added to a glass bottle, added with 10 ml of water at room temperature and shaken fully, placed on a magnetic stirrer and stirred overnight, and centrifuged for solid separation; 10 mg of sample was taken, heated to 220° C. at a speed of 10° C./min, and stayed at 220° C. for 2 min; and the solid was taken, and subjected to XRPD test to obtain the crystal form DCIII.

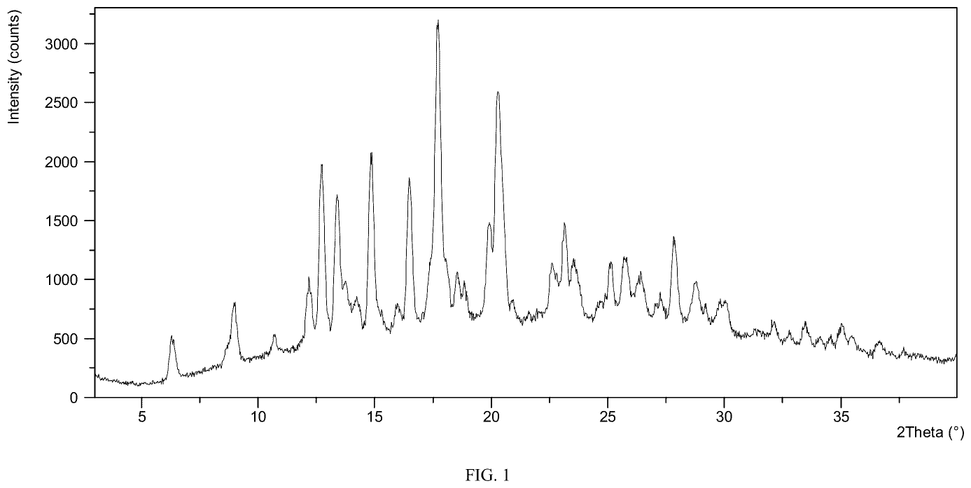

An XRPD diagram of the crystal form DCIII obtained according to Example 1a was shown in FIG. 1. XRPD data was shown in Table 1.

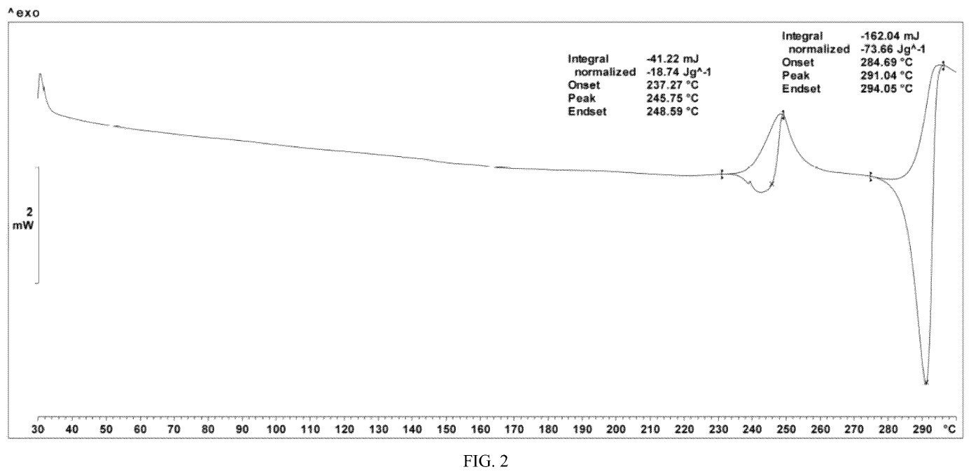

A DSC diagram of the crystal form DCIII obtained according to Example 1a was shown in FIG. 2.

TABLE 1

| Diffraction | | |
|---|---|---|
| angle | | Relative |
| 2theta | d value | intensity % |
| 6.30 | 14.03 | 13.23 |
| 8.96 | 9.87 | 22.51 |
| 10.71 | 8.26 | 13.04 |
| 12.20 | 7.26 | 28.27 |
| 12.65 | 7.00 | 52.51 |
| 13.39 | 6.61 | 54.25 |
| 14.22 | 6.23 | 23.18 |
| 14.83 | 5.97 | 66.03 |
| 15.95 | 5.56 | 20.45 |
| 16.49 | 5.37 | 57.38 |
| 17.76 | 4.99 | 100.00 |
| 18.54 | 4.79 | 29.72 |
| 18.87 | 4.70 | 25.46 |
| 19.85 | 4.47 | 40.44 |
| 20.25 | 4.39 | 80.47 |
| 22.61 | 3.93 | 31.45 |
| 23.15 | 3.84 | 43.57 |
| 23.52 | 3.78 | 31.56 |
| 25.13 | 3.54 | 30.31 |
| 25.68 | 3.47 | 31.44 |
| 26.47 | 3.37 | 25.12 |
| 27.22 | 3.28 | 19.31 |
| 27.84 | 3.21 | 37.86 |
| 28.76 | 3.10 | 24.07 |
| 30.10 | 2.97 | 17.69 |
| 32.11 | 2.79 | 11.10 |
| 32.81 | 2.73 | 7.63 |
| 33.44 | 2.68 | 9.92 |
| 35.07 | 2.56 | 9.73 |
| 36.69 | 2.45 | 4.11 |

### Example 1b

1.665 g of compound I was weighed and added to a 100 ml glass bottle; 13 ml of n-butanol was taken with a pipette to dissolve a sample; 20 ml of methyl tert-butyl ether was added dropwise to the glass bottle at room temperature; the glass bottle was placed in a 5° C. environment and stirred for 3 days; and a solid is separated and then dried at 30° C. XRPD test was carried out, and the result showed that the resulting product was the crystal form DCIII shown in the present invention.

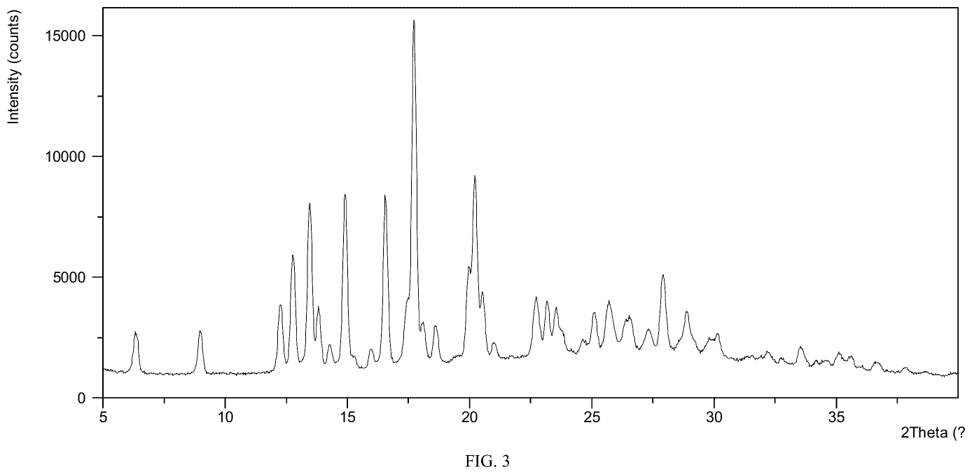

An XRPD diagram of the crystal form DCIII obtained according to Example 1b was shown in FIG. 3. XRPD data was shown in Table 2.

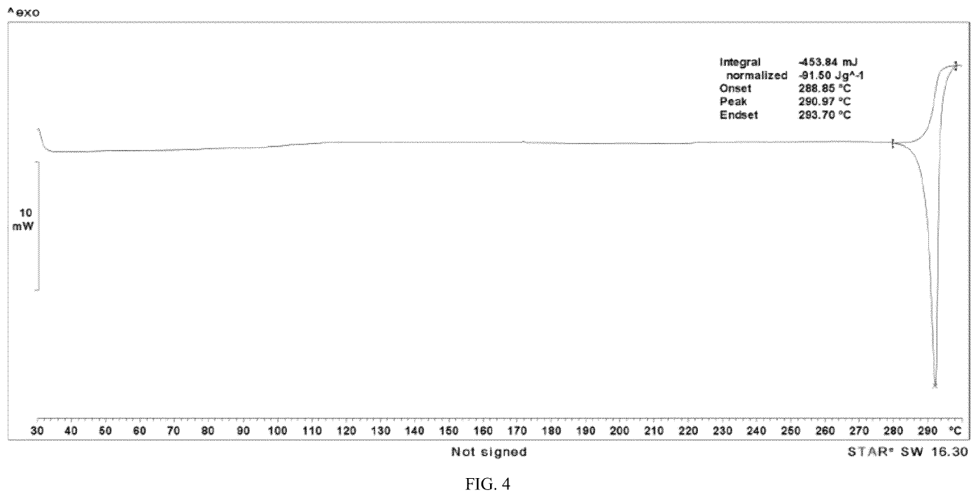

A DSC diagram of the crystal form DCIII obtained according to Example 1b was shown in FIG. 4.

TABLE 2

| Diffraction | | |
|---|---|---|
| angle | | Relative |
| 2theta | d value | intensity % |
| 6.30 | 14.02 | 10.12 |
| 9.00 | 9.83 | 12.12 |
| 12.31 | 7.19 | 18.07 |
| 12.74 | 6.95 | 31.64 |
| 13.43 | 6.59 | 47.39 |
| 13.80 | 6.42 | 18.08 |
| 14.26 | 6.21 | 8.20 |
| 14.90 | 5.95 | 50.73 |
| 15.96 | 5.55 | 6.95 |
| 16.53 | 5.36 | 47.47 |
| 17.40 | 5.10 | 19.42 |
| 17.71 | 5.01 | 100.00 |
| 18.12 | 4.90 | 14.49 |
| 18.56 | 4.78 | 13.09 |
| 19.93 | 4.45 | 28.45 |
| 20.22 | 4.39 | 56.28 |
| 20.53 | 4.33 | 23.11 |
| 21.00 | 4.23 | 8.86 |
| 22.74 | 3.91 | 21.30 |
| 23.16 | 3.84 | 20.75 |
| 23.54 | 3.78 | 19.14 |
| 24.61 | 3.62 | 9.66 |
| 25.09 | 3.55 | 17.59 |
| 25.62 | 3.48 | 18.66 |
| 26.35 | 3.38 | 14.91 |
| 26.59 | 3.35 | 15.80 |
| 27.32 | 3.26 | 12.70 |
| 27.87 | 3.20 | 26.84 |
| 28.85 | 3.09 | 17.76 |
| 30.16 | 2.96 | 11.60 |

### Example 1c

15 mg of compound I was weighed and added to a 3 ml glass bottle, and dissolved by taking 0.4 ml of n-butanol. 0.8 ml of n-hexane was slowly added dropwise to the glass bottle at room temperature, and the glass bottle was stirred at 5° C. for 1 day, followed by centrifugal separation. A solid was collected for XRPD test, and the result showed that the resulting product was the crystal form DCIII shown in the present invention.

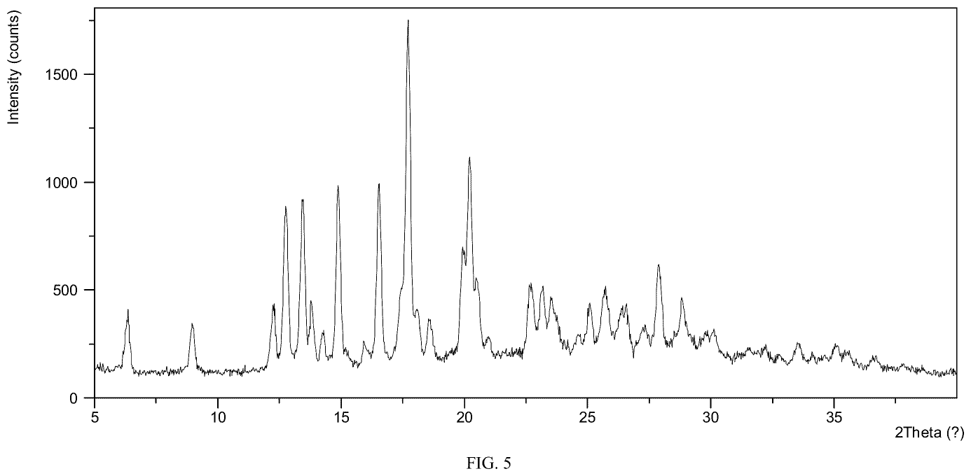

An XRPD diagram of the crystal form DCIII obtained according to Example 1c was shown in FIG. 5. XRPD data was shown in Table 3.

TABLE 3

| Diffraction | | |
|---|---|---|
| angle | | Relative |
| 2theta | d value | intensity % |
| 6.34 | 13.94 | 16.00 |
| 9.00 | 9.82 | 12.61 |
| 12.28 | 7.21 | 19.37 |
| 12.73 | 6.95 | 44.52 |
| 13.46 | 6.58 | 49.05 |
| 13.79 | 6.42 | 19.46 |
| 14.26 | 6.21 | 10.67 |
| 14.86 | 5.96 | 50.96 |
| 16.54 | 5.36 | 51.71 |
| 17.71 | 5.01 | 100.00 |
| 18.59 | 4.77 | 13.64 |
| 19.92 | 4.46 | 32.21 |
| 20.23 | 4.39 | 57.86 |
| 20.55 | 4.32 | 22.96 |
| 22.65 | 3.93 | 22.40 |
| 23.16 | 3.84 | 21.47 |
| 25.09 | 3.55 | 15.82 |
| 25.69 | 3.47 | 19.62 |
| 26.51 | 3.36 | 14.32 |
| 27.29 | 3.27 | 9.30 |
| 27.89 | 3.20 | 27.73 |
| 28.84 | 3.10 | 17.48 |
| 29.99 | 2.98 | 6.67 |

### Example 2: Dynamic Solubility of Crystal Form DCIII

When a drug solubility test was performed to predict the performances of a drug in vivo, it is important to simulate in vivo conditions as much as possible. For oral drugs, simulated gastric fluid (SGF), fasted state simulated intestinal fluid (FaSSIF) and fed state simulated intestinal fluid (FeSSIF) may be used to simulate in vivo conditions and predict a feeding effect, and the solubility tested in such media was closer to the solubility in the human body environment.

About 20 mg of crystal form I (prepared according to the disclosed method) reported in the US20200369662A1 and about 20 mg of crystal form DCIII of the present invention were respectively suspended in 1.5 mL of SGF, 1.5 mL of FeSSIF, 1.5 mL of FaSSIF and 1.5 mL of water to prepare suspensions, and equilibrated for 1 h, 4 h and 24 h; and then the contents of samples in the solutions were tested by high performance liquid chromatography respectively. The experimental results were shown in Table 4 and Table 5 below.

TABLE 4

| | Solubility (mg/mL) | | | | | |
|---|---|---|---|---|---|---|
| | SGF | | | FaSSIF | | |
| Onset crystal form | 1 h | 4 h | 24 h | 1 h | 4 h | 24 h |
| Anhydrous crystal form I | 3.77 | 3.86 | 3.11 | 0.02 | 0.01 | 0.03 |
| reported in | | | | | | |
| US20200369662A1 | | | | | | |
| Crystal form DCIII of the | 7.27 | 6.24 | 4.54 | 0.05 | 0.04 | 0.04 |
| present invention | | | | | | |

TABLE 5

| | Solubility (mg/mL) | | | | | |
|---|---|---|---|---|---|---|
| | FeSSIF | | | H₂O | | |
| Onset crystal form | 1 h | 4 h | 24 h | 1 h | 4 h | 24 h |
| Anhydrous crystal form I | 0.03 | 0.04 | 0.06 | 0.01 | 0.01 | 0.01 |
| reported in | | | | | | |
| US20200369662A1 | | | | | | |
| Crystal form DCIII of the | 0.15 | 0.2 | 0.08 | 0.03 | 0.03 | 0.03 |
| present invention | | | | | | |

Experimental results of the dynamic solubility indicated that: compared to the anhydrous crystal form I reported in US20200369662A1, the crystal form DCIII of the present invention had higher solubility in SGF, FaSSIF, FeSSIF and pure water.

### Example 3: Stability of Crystal Form DCIII

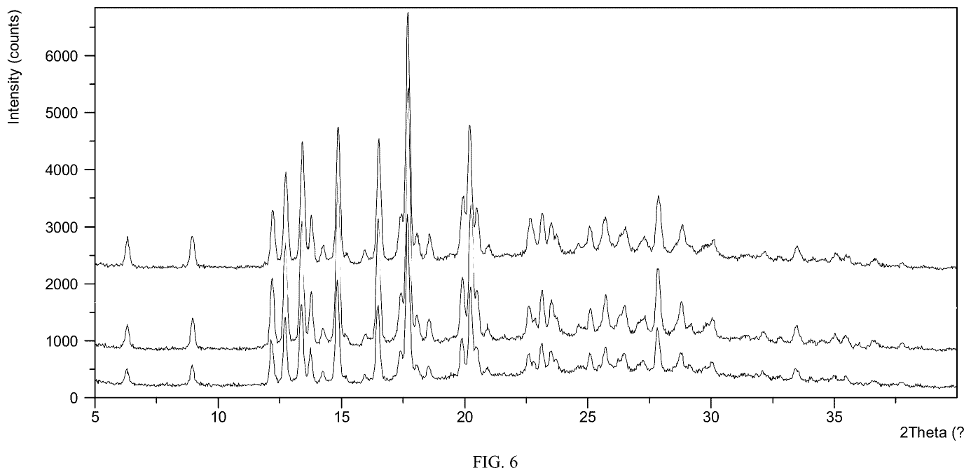

About 5 mg of crystal form DCIII prepared in the present invention was weighed, sealed with an aluminum foil bag, and placed under the conditions of 25° C./60 RH, 40° C./75% RH, and 60° C./75% RH respectively; and the purity and crystal form were measured by HPLC and XRPD. The experimental results were shown in Table 6. An XRPD overlay was shown in FIG. 6.

TABLE 6

| Condition of | Time of | | |
|---|---|---|---|
| placement | placement | Crystal form | Purity |
| Onset | — | Crystal form DCIII | 99.44% |
| 25° C./60% RH | 1 month | Crystal form DCIII | 99.45% |
| 40° C./75% RH | 1 month | Crystal form DCIII | 99.49% |
| 60° C./75% RH | 1 month | Crystal form DCIII | 99.50% |

The results showed that the crystal form DCIII of the present invention could maintain the physicochemical stability for at least 1 month under three conditions: 25° C./60% RH, 40° C./75% RH, and 60° C./75% RH.

### Example 4: Mechanical Stability of Crystal Form DCIII

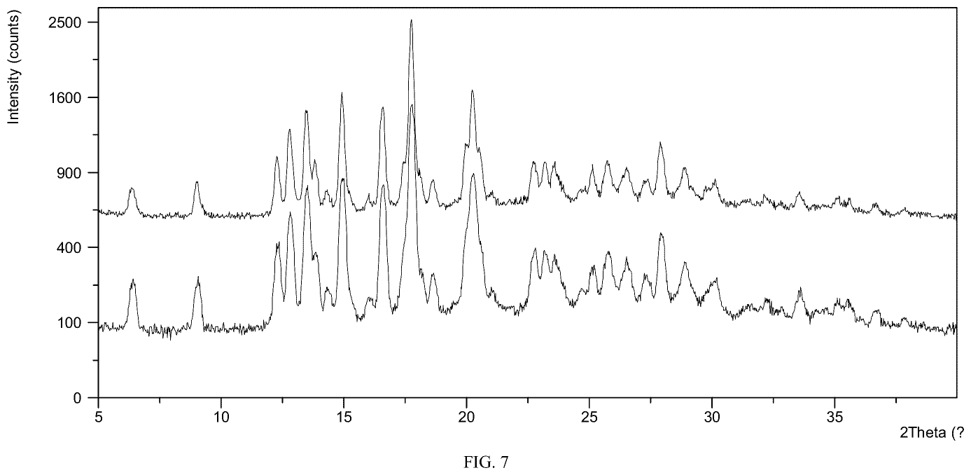

The crystal form DCIII was placed in a mortar and ground manually for 5 min, and an XRPD test was performed before and after grinding. The XRPD comparison before and after grinding was shown in FIG. 7. The results showed that the crystal form DCIII of the present invention remained unchanged before and after grinding, and no significant decrease in the crystallinity was observed, indicating that the crystal form DCIII had good mechanical stability.

### Example 5: Hygroscopicity of Crystal Form DCIII

About 100 mg of the crystal form DCIII of the present invention was weighed, and placed at 25±1° C., 80% relative humidity for 24 h, and the mass of a sample was recorded before and after placement. The specific results were shown in Table 7.

According to the description of hygroscopicity characteristics and the definition (9103 Drug Hygroscopicity Experiment Guiding Principles, 2020 edition of General Principles, Chinese Pharmacopoeia, experimental conditions: 25±1° C., 80% relative humidity) of the hygroscopic weight gain:
deliquescent: enough water is absorbed to form liquid
very hygroscopic: the hygroscopic weight gain is not less than 15.0%
hygroscopic: the hygroscopic weight gain is less than 15.0% but not less than 2.0%
slightly hygroscopic: the hygroscopic weight gain is less than 2.0% but not less than 0.2%
no or almost no hygroscopicity: the hygroscopic weight gain is not less than 0.2%.

TABLE 7

| Onset | Condition | | Mass after | Weight | Weight |
|---|---|---|---|---|---|
| mass | of | Time of | placement | gain | gain |
| (mg) | placement | placement | (mg) | mass (mg) | percentage |
| 102.76 | 25 ± 1° C., | 24 h | 103.40 | 0.64 | 0.6% |
| | 80% RH | | | | |

The results showed that the crystal form DCIII of the present invention was slightly hygroscopic, and had small hygroscopicity, which indicated that the crystal DCIII was not easily deliquescent in the process of drug production and storage.

### Example 6: Purification Effect of Crystal Form DCIII

30 mg of sample with a purity of 98.4% was weighed, added with 0.4 ml of n-butanol to dissolve and clarify, slowly added with 2 ml of methyl tert-butyl ether, then placed at 5° C. and stirred overnight; a solid was tested to be the crystal form DCIII. HPLC results showed that the purity was 99.1%, indicating that the crystal form DCIII had good purification and impurity removal effects, and had great benefits in the process of industrial production and amplification.

The above examples are only to illustrate the technical conception and characteristics of the present invention, and are intended to enable those skilled in the art to understand the content of the present invention and to implement it accordingly, but not intended to limit the protection scope of the present invention. The equivalent changes or modifications made according to the spiritual essence of the present invention shall be covered by the protection scope of the present invention.

What is claimed is:

1. A crystal form DCIII of a compound I, 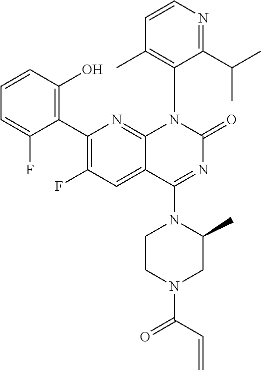 [SMILES: C=CC(=O)N1CCN(c2nc(=O)n(-c3c(C)ccnc3C(C)C)c3nc(-c4c(O)cccc4F)c(F)cc23)[C@@H](C)C1]
wherein with a use of Cu—Kα radiation, a first characteristic peak appears at each diffraction angles 2theta having values of 6.3°+0.2°, 9.0°+0.2°, 14.8°+0.2°, 17.8°+0.2°, 12.7°+0.2°, 16.5°+0.2°, 12.2°+0.2°, 13.4°±0.2°, and 20.3°+0.2° in an X-ray powder diffraction pattern.

2. The crystal form DCIII of the compound I according to claim 1, wherein with the use of the Cu—Kα radiation, a second characteristic peak appears at one or two of diffraction angles 2theta having values of 23.2°±0.2° and 27.8°+0.2° in the X-ray powder diffraction pattern.

3. A preparation method of the crystal form DCIII of the compound I according to claim 1, comprising: weighing a predetermined amount of the compound I; adding a predetermined amount of water to the compound I to form a suspension; stirring the suspension for a period of time; separating a solid from the suspension, and heating the solid to a temperature of 190° C.-235° C.; and collecting a heated solid to obtain the crystal form DCIII of the compound I.

4. A preparation method of the crystal form DCIII of the compound I according to claim 1, comprising: weighing a predetermined amount of the compound I; dissolving the compound I with predetermined amount of n-butanol to obtain a first resulting solution, then adding methyl tert-butyl ether or n-hexane to the first resulting solution to obtain a second resulting solution, stirring the second resulting solution at 5° C.±2° C., and performing a recrystallization on the second resulting solution to obtain a recrystallized product; and centrifuging the recrystallized product for a solid separation to obtain the crystal form DCIII of the compound I.

5. A pharmaceutical composition, comprising a therapeutically effective amount of the crystal form DCIII of the compound I according to claim 1, and a pharmaceutically acceptable carrier or excipient.

6. A method of preparing a KRAS G12C inhibitor drug, comprising using the crystal form DCIII of the compound I according to claim 1.

7. A method of preparing a drug for treating diseases caused by a KRAS G12C gene mutation, comprising using the crystal form DCIII of the compound I according to claim 1.

8. (canceled)

9. (canceled)

10. (canceled)
